(19) United States
(12) Patent Application Publication
(10) Pub. No.: US 2007/0191315 A1
(43) Pub. Date: Aug. 16, 2007

(54) Method for administering ibandronate

(76) Inventor: Bengt Bergstrom, Paramus, NJ (US)

Correspondence Address: HOFFMANN-LA ROCHE INC.;PATENT LAW DEPARTMENT, 340 KINGSLAND STREET, NUTLEY, NJ, 07110, US

(21) Appl. No.: 11/655,795

(22) Filed: Jan. 22, 2007

Related U.S. Application Data
(60) Provisional application No. 60/773,902, filed on Feb. 16, 2006.

(51) Int. Cl. A61K 31/66

(52) U.S. Cl. 514/102

(57) ABSTRACT

Disclosed is an improved method for treating pain associated with metastatic bone disease comprising the quick administration of an IV loading dose of ibandronate followed by an oral or intravenous (IV) maintenance dosing regimen. This method provides desirable therapeutic effects, including fast onset of pain relief, with a tolerable level of toxicity and is preferable for patient compliance.

### PRIORITY TO RELATED APPLICATIONS

This application claims the benefit of U.S. Provisional Application No. 60/773,902, filed Feb. 16, 2006, which is hereby incorporated by reference in its entirety.

### FIELD OF THE INVENTION

The present invention is directed to an improved method of administration of ibandronate in the treatment of bone disorders, in particular the pain that typically accompanies metastatic bone disease. Specifically, the invention relates to an improved method for the treatment of bone pain associated with metastatic bone disease comprising the rapid administration of IV loading doses of ibandronate followed by an oral maintenance dosing regimen.

### BACKGROUND OF THE INVENTION

The relationship between malignancy and pain is long established, and pain is often the first symptom of malignancy in newly diagnosed patients, as well as the herald of recurrence or progression in patients receiving antineoplastic treatment.

Bone is a common site of metastatic disease for commonly occurring malignancies: 30% -40% of patients with lung cancer, 65-75% of patients with breast cancer and 65% -75% of patients with prostate cancer develop bone metastases. Given the worldwide incidence of these neoplasms, bone metastases represent a large public health problem. Pain associated with osseous metastases is quite common and can be particularly incapacitating. In a recent review, the incidence of bone pain at the time of diagnosis of bone involvement was 63% in patients with multiple myeloma; 71% in patients with breast cancer; 42% of patients with prostate cancer; 62% in patients with lung cancer; 58% in patients with renal cell cancer; and between 37% and 94% in patients with a variety of other solid tumors and bone metastases. In one study, the incidence of pain in patients with bone metastases secondary to lung, breast or other cancers was 80%, 77% or 67%, respectively. In other surveys, bone pain has been cited as the primary cause of pain among cancer patients, occurring in 38% of all cancer patients, and experienced as severe in 39% of these patients. Other studies have demonstrated a high incidence of pain in patients with malignancies that have a propensity to disseminate to bone, and in a third of these patients, the pain was considered by the patients to be moderate to severe in intensity.

The presence of bone pain is not related to type of tumor, location, or number and size of metastases. Pain may be secondary to microfractures in bone trabeculae; stretching of periosteum by expanding tumor, or nerve entrapment by tumor. Osteolytic destruction of bone is mediated by many of the same cytokines and growth factors that stimulate peripheral sensory receptors in bone, and this may be another cause of bone pain in patients with bone metastases. This latter point may also explain why pain produced by bone metastases is disproportionate to the size of metastases or the degree of bone involvement. Clearly, pain secondary to bone metastases affects large numbers of patients worldwide and remains, despite better education of health care professionals regarding the treatment of pain, a vastly unsatisfied medical need.

In response to this long-felt need, the present invention provides methods for the relief of pain associated with metastatic bone disease comprising administration of an effective amount of ibandronic acid or a pharmaceutically acceptable salt thereof to a subject in need of treatment.

Ibandronic acid is a nitrogen containing bisphosphonate. The chemical name for ibandronate is 3-(N-methyl-N-pentyl)amino-1-hydroxypropane-1,1-diphosphonic acid. Ibandronic acid may be represented by the following formula:

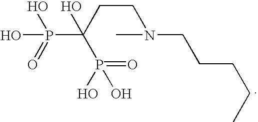

Ibandronate, which is the sodium salt monohydrate of ibandronic acid, has the molecular formula C₉H₂₂NO₇P₂Na.H₂0 and a molecular weight of 359.24. Ibandronate and its salts are disclosed and claimed in U.S. Pat. No. 4,927,814, which disclosure is incorporated herein by reference.

Ibandronate is currently approved in the European Union (EU), under the trade name of Bondronat® for prevention of skeletal events in patients with breast cancer and bone metastases, and hypercalcemia of malignancy, as both an IV and oral formulation. As of the present filing date, Bondronate is not approved for use in oncology in the U.S., although the same active ingredient is approved for the treatment and prevention of post-menopausal osteoporosis under the trade-name of Boniva®.

Pain associated with metastatic bone disease is a major issue in the treatment of patients afflicted with this condition. Quick onset of effect, and thereby of pain relief, is desirable. Oral treatment is substantially slower in onset than intravenous therapy. On the other hand, oral therapy is more convenient for the patient.

### SUMMARY OF THE INVENTION

A new protocol for the treatment of pain associated with bone metastases is disclosed that provides desirable therapeutic effects, including fast onset of pain relief, with minimal adverse events and is preferable for patient convenience and compliance.

In one embodiment, the present invention relates to a method of alleviating or treating the pain associated with metastatic bone disease in a subject in need thereof comprising the steps of (a) administering intravenously a loading dose of ibandronate selected from about 4 mg to about 25 mg, over a loading period of from 1 to 3 days; (b) followed by orally administering a daily maintenance dose of ibandronate, the maintenance dose being administered commencing on from the 3^{rd} to about the 30^{th} day after the first day of the loading period.

In another embodiment, the present invention relates to a method of alleviating or treating pain associated with metastatic bone disease in a subject in need thereof comprising the steps of
(a) administering intravenously a loading dose of ibandronate selected from about 4 mg to about 25 mg, over a loading period of from 1 to 3 days;
(b) followed by orally administering a daily maintenance dose of ibandronate, the maintenance dose being administered commencing on from the 21^{st} to about the 30^{th} day after the first day of the loading period.

### DETAILED DESCRIPTION OF THE INVENTION

All patents, patent applications, and other publications cited herein are in incorporated by reference in their entirety. In the event of a conflict, the present specification controls.

As used herein, the following terms have the given meanings:

“Administering” means oral or intravenous administration.

“Loading dose” mean the dose that is initially administered to a patient. The dose is effective to product the desired therapeutic effect or response when administered over the loading period. Thus, the loading dose of ibandronate is a bone resorption-inhibiting and pain-reducing amount. Bone resorption inhibition can be measured by biomarker or bone biopsy, BMD, micro-computerized tomography (micro-CT), peripheral-CT, or another technique.

“Loading period” means that period of one, two, or three consecutive days during which the loading dose is administered to a patient.

“Bone resorption inhibition” means slowing or inhibiting the progression of, or treating or preventing bone resorption by the direct or indirect alteration of osteoclast formation or activation. Thus, inhibition of bone resorption refers to slowing, treating or preventing bone loss.

“Continuous administration” or “continuous dosing schedule” means the dosing is repeated until the desired therapeutic effect is obtained. This is in contrast to cyclical or intermittent administration.

“Maintenance dose” means the dose given to a subject following the loading period. The maintenance dose is such as to be a therapeutic amount.

“Maintenance period” is the period of time following the loading period and during which the subject is given, continuously, a dose of ibandronate.

“Metastatic bone disease” means a tumor localized in a bone that has its origin in another body tissue, e.g., breast or prostate.

“Metastatic bone pain” means pain caused by metastatic bone disease.

“Mild renal impairment” means that a patient has a baseline creatinine clearance level of between 51 and 80 mL/min.

“Moderate renal impairment” means that a patient has a baseline creatinine level of between 30 and 50 mL/min.

“Normal renal function” means that a patient has a baseline creatinine level of 80 mL/min or above.

“Therapeutic amount” “or therapeutically effective amount” means an amount of ibandronate sufficient to ameliorate or relieve the symptoms and/or condition to be treated.

The present invention relates to a method of treating the pain associated with metastatic bone disease in a subject in need thereof comprising the steps of

(a) administering intravenously a loading dose of ibandronate selected from about 4 mg to about 25 mg, over a loading period of from 1 to 3 days;

(b) followed by orally administering a daily maintenance dose of ibandronate, the maintenance dose being administered commencing on from the 3^{rd} to about the 30^{th} day after the first day of the loading period.

Preferably, said oral maintenance dose will be at least twice the intravenous loading dose.

Preferably, the loading dose in the methods described herein is about 18 mg of ibandronate, which most preferably is administered in equal doses over a loading period of 3 consecutive days. The loading dose can also be, e.g., 9 or 12 mg of ibandronate administered in equal doses over a loading period of 3 consecutive days, depending on the health of the patient. Preferably each daily administration of the loading dose takes from about 15 to about 60 minutes, most preferably about 15 minutes.

In another embodiment, the invention relates to a method for treating metastatic bone pain in a patient, comprising the steps of:

(a) assessing the renal function of the patient to determine if the patient is suffering from mild to moderate renal impairment; and

(b) if the patient has normal renal function, administering intravenously a loading dose of ibandronate of about 18 mg, in equal daily doses over a loading period of 3 consecutive days, followed by orally administering a 50 mg daily maintenance dose of ibandronate, administration of the maintenance dose starting 21 to 30 days after the first day of the loading period; and

(c) if the patient is suffering from mild or moderate renal impairment, administering intravenously a loading dose of ibandronate of about 9 to about 12 mg, in equal daily doses over a loading period of 3 consecutive days, followed by orally administering a 50 mg daily maintenance dose of ibandronate, administration of the maintenance dose starting 21 to 30 days after the first day of the loading period.

In another embodiment, the invention relates to a method for treating metastatic bone pain in a patient, comprising the steps of:

(a) assessing the renal function of the patient to determine if the patient is suffering from mild or moderate renal impairment; and

(b) if the patient has normal renal function, administering intravenously a loading dose of ibandronate of about 18 mg, in equal daily doses over a loading period of 3 consecutive days, followed by orally administering a daily 50 mg maintenance dose of ibandronate, administration of the maintenance dose starting 21 to 30 days after the first day of the loading period; and

(c) if the patient is suffering from mild renal impairment, administering intravenously a loading dose of ibandronate of about 12 mg, in equal daily doses over a loading period of 3 consecutive days, followed by orally administering a daily 50 mg maintenance dose of ibandronate, administration of the maintenance dose starting 21 to 30 days after the first day of the loading period; and

(d) if the patient is suffering from moderate renal impairment, administering intravenously a loading dose of ibandronate of about 9 mg, in equal daily doses over a loading period of 3 consecutive days, followed by orally administering a 50 mg daily maintenance dose of ibandronate, administration of the maintenance dose starting 21 to 30 days after the first day of the loading period.

In another embodiment, the invention relates to a method for treating metastatic bone pain in a patient, comprising the steps of:

(a) assessing the renal function of the patient to determine if the patient is suffering from mild or moderate renal impairment; and

(b) if the patient has normal renal function or is suffering from mild renal impairment, administering intravenously a loading dose of ibandronate of about 18 mg, in equal daily doses over a loading period of 3 consecutive days, followed by orally administering a daily maintenance dose of ibandronate, administration of the maintenance dose starting 21 to 30 days after the first day of the loading period; and

(c) if the patient is suffering from moderate renal impairment, administering intravenously a loading dose of ibandronate of about 12 mg, in equal daily doses over a loading period of 3 consecutive days, followed by orally administering a daily maintenance dose of ibandronate, administration of the maintenance dose starting 21 to 30 days after the first day of the loading period.

Preferably the oral maintenance dose of ibandronate is about 50 mg. Most preferably administration of the maintenance dose commences about 21 days after the first day of the loading period and continues daily thereafter.

The methods of the present invention comprise use of pharmaceutical compositions that contain a therapeutically effective amount of ibandronate, a known and commercialized bisphosphonate. These compositions typically also comprise excipients. If the composition is oral, such excipients can include binders (e.g., polyvinylpyrrolidone (e.g. Povidone®) or hydroxypropylmethyl cellulose (e.g., Pharmacoat®)), fillers (e.g. lactose in hydrate or anhydrate form, cellulose in microcrystalline or fibrous form (e.g. Avicel®) or starch), disintegrants (e.g., cross-linked polyvinyl pyrrolidone (e.g. Crospovidone® USPNF) or cross carmelose), lubricants (e.g., stearic acid or magnesium stearate), and flow-regulators (e.g. colloidal silicon dioxide). If the composition is parenteral, such excipients include polyethylene glycol and chelating agents such as a polyacetic acid or a pharmaceutically acceptable salt thereof like ethylene diamine tetraacetic acid (EDTA), diethylenetriaminepentaacetic acid (DTPA), ethyleneglycol-bis(beta-aminoethyl ether)-tetraacetid acid (EGTA), citric acid, tartaric acid, phosphonic acid, etc.

If the ibandronate is oral, the preferred form is a film coated tablet. Film coatings for tablets are known in the art.

Various compositions and formulations of ibandronate are known. See, e.g., U.S. Pat. No. 4,927,814; U.S. Pat. No. 5,662,918; U.S. Pat. No. 6,143,326; U.S. Pat. No. 6,294,196; U.S. Pat. No. 6,677,320; and US 2004/0121007 A1, all of which, to the extent necessary, are herein incorporated by reference. Processes for making ibandronate-containing pharmaceutical compositions are also known. See, e.g., U.S. Pat. No. 6,419,955 and U.S. Pat. No. 6,627,221.

Reference to a specific weight or percentage of ibandronate in the present invention is on an acid active weight bases, unless otherwise indicated.

### EXAMPLES

The following examples further describe embodiments within the scope of the present invention. The examples are presented for purposes of demonstrating, but not limiting, the invention.

### Example 1

A multicenter, randomized, active-controlled, double-blind, parallel groups trial is conducted. Patients are centrally randomized via an interactive voice response system (IVRS) to treatment either with ibandronic acid or zoledronic acid. Study treatment begins within 96 hours of randomization. Treatment continues for 24 weeks. Patients record their pain on the WORST PAIN and the AVERAGE PAIN scales of the BPI, as well as daily analgesic use, on a nightly basis. Patients record the degree to which their pain interferes with normal functioning and any opioid side-effects weekly. WHO Performance Status, quality of life based on the EORTC QLQ-C30 instrument and quality of life based on the FACT-BP, an instrument specifically designed to measure quality of life in patients with bone pain are recorded at Day 8 and Weeks 6, 12, 18 and 24 of the trial. Patients have skeletal surveys performed at Baseline and at Weeks 12 and 24 as part of the monitoring of SREs.

Patients randomized to the ibandronic acid arm receive ibandronic acid 6 mg intravenously over 15 minutes on Days 1, 2 and 3 of the trial, followed by ibandronic acid 50 mg orally daily beginning on Day 22 of the study. Patients also receive a placebo infusion every 3-4 weeks, depending on the underlying chemotherapy regimens patients may be receiving. Patients who are not receiving chemotherapy receive placebo infusions every 3 weeks. Intravenous ibandronic acid is diluted with 100 mL 0.9% sodium chloride or 5% dextrose solution.

Patients randomized to the zoledronic acid arm receive a scheduled dose of zoledronic acid intravenously over 15 minutes on Day 1 and a placebo infusion intravenously over 15 minutes on Days 2 and 3, followed by the scheduled dose zoledronic acid over 15 minutes every 3-4 weeks, depending on the underlying chemotherapy regimens patients may be receiving. Patients who are not receiving chemotherapy are treated with zoledronic acid every 3 weeks. Patients also receive oral placebo daily beginning on Day 22 of the study. Zoledronic acid is diluted with 100 mL 0.9% sodium chloride or 5% dextrose solution. All patients, regardless of treatment assignment, also receive calcium supplementation, 500 mg orally and Vitamin D, 400 IU orally, daily. The calcium and Vitamin D supplementation are administered in the evening.

Patients on the ibandronate regimen show significantly lower pain scores compared to patients receiving placebo treatment.

What is claimed is:

1. A method of treating the pain associated with metastatic bone disease in a subject in need thereof comprising the steps of:
(a) intravenously administering a loading dose of ibandronate of about 4 mg to about 25 mg, over a loading period of from 1 to 3 days; then
(b) orally administering a daily maintenance dose of ibandronate, the maintenance dose being administered commencing on from the 3^{rd} to about the 30^{th} day after commencement of the loading period.

2. The method of claim 1 wherein said loading dose is about 18 mg.

3. The method of claim 2 wherein three portions of said loading dose are administered in equal amounts over a loading period of three consecutive days.

4. The method of claim 3 wherein said three portions of said loading dose are administered over about a period of from about 15 to about 60 minutes per day on each of said three consecutive days.

5. The method of claim 4 wherein said loading dose is administered over a period of about 15 minutes per day on each of said three consecutive days.

6. The method of claim 5 wherein the daily maintenance dose of ibandronate is about 50 mg per day, commencing on about the 21^{st} day after the first day of the loading period.

7. A method for treating metastatic bone pain in a patient, comprising the steps of:
(a) assessing the renal function of the patient to determine if the patient is suffering from mild to moderate renal impairment; and
(b) if the patient has normal renal function, administering intravenously a loading dose of ibandronate of about 18 mg, in equal daily doses over a loading period of 3 consecutive days, followed by orally administering a 50 mg daily maintenance dose of ibandronate, administration of the maintenance dose starting 21 to 30 days after the first day of the loading period; and
(c) if the patient is suffering from mild or moderate renal impairment, administering intravenously a loading dose of ibandronate of about 9 to about 12 mg, in equal daily doses over a loading period of 3 consecutive days, followed by orally administering a 50 mg daily maintenance dose of ibandronate, administration of the maintenance dose starting 21 to 30 days after the first day of the loading period

8. A method for treating metastatic bone pain in a patient, comprising the steps of:
(a) assessing the renal function of the patient to determine if the patient is suffering from mild or moderate renal impairment; and
(b) if the patient has normal renal function, administering intravenously a loading dose of ibandronate of about 18 mg, in equal daily doses over a loading period of 3 consecutive days, followed by orally administering a daily 50 mg maintenance dose of ibandronate, administration of the maintenance dose starting 21 to 30 days after the first day of the loading period; and
(c) if the patient is suffering from mild renal impairment, administering intravenously a loading dose of ibandronate of about 12 mg, in equal daily doses over a loading period of 3 consecutive days, followed by orally administering a daily 50 mg maintenance dose of ibandronate, administration of the maintenance dose starting 21 to 30 days after the first day of the loading period; and
(d) if the patient is suffering from moderate renal impairment, administering intravenously a loading dose of ibandronate of about 9 mg, in equal daily doses over a loading period of 3 consecutive days, followed by orally administering a 50 mg daily maintenance dose of ibandronate, administration of the maintenance dose starting 21 to 30 days after the first day of the loading period.

9. A method for treating metastatic bone pain in a patient, comprising the steps of:
(a) assessing the renal function of the patient to determine if the patient is suffering from mild or moderate renal impairment; and
(b) if the patient has normal renal function or is suffering from mild renal impairment, administering intravenously a loading dose of ibandronate of about 18 mg, in equal daily doses over a loading period of 3 consecutive days, followed by orally administering a daily maintenance dose of ibandronate, administration of the maintenance dose starting 21 to 30 days after the first day of the loading period; and
(c) if the patient is suffering from moderate renal impairment, administering intravenously a loading dose of ibandronate of about 12 mg, in equal daily doses over a loading period of 3 consecutive days, followed by orally administering a daily maintenance dose of ibandronate, administration of the maintenance dose starting 21 to 30 days after the first day of the loading period.
